(19) United States
(12) Patent Application Publication
(10) Pub. No.: US 2010/0076096 A1
(43) Pub. Date: Mar. 25, 2010

(54) PROCESS FOR THE PREPARATION OF ALKYLATE AND MIDDLE DISTILLATE

(76) Inventors: Hans Peter Alexander Calis, The Hague (NL); Georghios Agamemnonons Hadjigeorge, Sugar Land, TX (US); Weijian Mo, Sugar Land, TX (US); Easwar Santhosh Ranganathan, Houston, TX (US)

Correspondence Address: SHELL OIL COMPANY, P O BOX 2463, HOUSTON, TX, 772522463, US

(21) Appl. No.: 12/525,690

(22) PCT Filed: Feb. 4, 2008
(86) PCT No.: PCT/EP2008/051311

§ 371 Date: Aug. 4, 2009

(30) Foreign Application Priority Data
Feb. 6, 2007 (EP) 07101832.9

(51) Int. Cl. C07C 27/06; C10G 57/00

(52) U.S. Cl. 518/702; 208/67

(57) ABSTRACT

A process for the preparation of alkylate and middle distillate, the process comprising: (a) catalytically cracking a first hydrocarbon feedstock by contacting the feedstock with a cracking catalyst comprising a shape-selective additive at a temperature in the range of from 450 to 650° C. within a riser or downcomer reaction zone to yield a first cracked product comprising middle distillate and a spent cracking catalyst; (b) regenerating the spent cracking catalyst to yield a regenerated cracking catalyst; (c) contacting, within a second reaction zone, at least part of the regenerated cracking catalyst obtained in step (b) with a second hydrocarbon feedstock at a temperature in the range of from 500 to 800° C. to yield a second cracked product and a used regenerated catalyst, the second feedstock comprising at least 70 wt % C₅₊ hydrocarbons obtained in a Fischer-Tropsch hydrocarbon synthesis process; (d) using the used regenerated catalyst as at least part of the cracking catalyst in step (a); and (e) alkylating at least a portion of the second cracked product in an alkylation unit to obtain alkylate.

### FIELD OF THE INVENTION

This invention provides a process for the preparation of alkylate and middle distillate.

### BACKGROUND OF THE INVENTION

Fluidised catalytic cracking (FCC) of heavy hydrocarbons to produce lower boiling hydrocarbon products such as gasoline is well known in the art. FCC processes have been around since the 1940's. Typically, an FCC unit or process includes a riser reactor, a catalyst separator and stripper, and a regenerator. A hydrocarbon feedstock, typically heavy vacuum distillates or residuum of crude oil distillation, is introduced into the riser reactor wherein it is contacted with hot FCC catalyst from the regenerator. The mixture of the feedstock and FCC catalyst passes through the riser reactor and into the catalyst separator wherein the cracked product is separated from the FCC catalyst. The separated cracked product passes from the catalyst separator to a downstream separation system and the separated catalyst passes to the regenerator where the coke deposited on the FCC catalyst during the cracking reaction is burned off the catalyst to provide a regenerated catalyst. The resulting regenerated catalyst is used as the aforementioned hot FCC catalyst and is mixed with fresh hydrocarbon feedstock that is introduced into the riser reactor.

Many FCC processes and systems are designed so as to provide for a high conversion of the FCC feedstock to products having boiling temperatures in the gasoline boiling range. There are situations, however, when it is desirable to provide for the high conversion of the FCC feedstock to middle distillate boiling range products, as opposed to gasoline boiling range products, and to lower olefins. However, making lower olefins requires high severity and high reaction temperature reaction conditions. These conditions normally result in low middle distillate product yield and poor middle distillate product quality. It is therefore very difficult in the conventional cracking of hydrocarbons to provide simultaneously for both a high yield of lower olefins and a high yield of middle distillate products.

In WO 2006/020547 is disclosed a process for the preparation of middle distillates and lower olefins, wherein a gasoil feedstock is contacted within a riser reactor with a middle distillate selective cracking catalyst to yield cracked products and spent cracking catalyst. The spent cracking catalyst is regenerated and a gasoline feedstock is contacted with the regenerated cracking catalyst in a dense bed reactor zone under high severity cracking conditions to yield cracked gasoline products including lower olefins and used regenerated cracking catalyst. The used regenerated cracking catalyst is utilised as the middle distillate selective cracking catalyst in the riser.

In the process of WO 2006/020547, two reactors and a single catalyst regenerator are used. Thus, coke deposited on the catalyst in two reactors has to be removed in a single catalyst regenerator. It will be necessary to control coke formation carefully in order to prevent build-up of coke in the system due to limited capacity of the regenerator.

In FIG. III of U.S. Pat. No. 3,928,172, a process arrangement with three reaction zones and a single catalyst regenerator is disclosed. Gasoline product of gasoil cracking is re-cracked in a dense fluid bed reaction zone using freshly regenerated catalyst; the catalyst used for gasoline re-cracking is then used for gasoil cracking in a riser reaction zone; and catalyst separated from the riser reaction zone is used in a third reaction zone for cracking virgin naphtha. In the process of U.S. Pat. No. 3,928,172, gasoline and alkylate are produced. The process of U.S. Pat. No. 3,928,172 does not produce middle distillates.

It is known that, apart from conventional FCC feedstocks such as vacuum gas oil (VGO) or residuum of atmospheric crude oil distillation, hydrocarbon streams produced by Fischer-Tropsch hydrocarbon synthesis can be used as feedstock for a FCC unit. A disadvantage, however, of the use of Fischer-Tropsch derived hydrocarbons is that the amount of coke deposited on the catalyst is typically insufficient to provide for the heat needed for the endothermic cracking reaction and therefore it is difficult to heat balance the process.

Several solutions have been proposed to solve the heat balance problem. In U.S. Pat. No. 4,684,756 for example is disclosed a process for fluidised catalytic cracking of a wax produced by Fischer-Tropsch hydrocarbon synthesis. It is mentioned addition of heat to the regeneration step is needed to heat balance the FCC operation. Synthesis gas and tail gases from the Fischer-Tropsch synthesis are mentioned as potential fuels sources for providing the additional heat.

### SUMMARY OF THE INVENTION

It has now been found that it is possible to heat balance a fluidised catalytic cracking process using a hydrocarbon feedstock obtained by a Fischer-Tropsch hydrocarbon synthesis process by using a line-up with two reactors and a single regenerator similar to the line-up as disclosed in WO 2006/020547. A feedstock obtained by Fischer-Tropsch hydrocarbon synthesis is contacted with a regenerated cracking catalyst in one reactor to produce cracked product comprising lower olefins and used regenerated catalyst. The used regenerated catalyst is used for cracking a further feedstock in a further reactor to produce cracked product comprising middle distillate and spent catalyst. The spent catalyst is regenerated to produce regenerated cracking catalyst to be used for cracking the feedstock obtained by Fischer-Tropsch hydrocarbon synthesis. The lower olefins obtained are alkylated in an alkylation unit to produce alkylate.

Accordingly, the invention provides a process for the preparation of alkylate and middle distillate, the process comprising:

(a) catalytically cracking a first hydrocarbon feedstock by contacting the feedstock with a cracking catalyst comprising a shape-selective additive at a temperature in the range of from 450 to 650° C. within a riser or downcomer reaction zone to yield a first cracked product comprising middle distillate and a spent cracking catalyst;
(b) regenerating the spent cracking catalyst to yield a regenerated cracking catalyst;
(c) contacting, within a second reaction zone, at least part of the regenerated cracking catalyst obtained in step (b) with a second hydrocarbon feedstock at a temperature in the range of from 500 to 800° C. to yield a second cracked product and a used regenerated catalyst, the second feedstock comprising at least 70 wt % C₅₊ hydrocarbons obtained in a Fischer-Tropsch hydrocarbon synthesis process;
(d) using the used regenerated catalyst as at least part of the cracking catalyst in step (a); and
(e) alkylating at least a portion of the second cracked product in an alkylation unit to obtain alkylate.

Thus, the invention provides a process to produce both middle distillate and considerable amounts of high octane compounds such as iso-paraffins, by alkylating unsaturated, cracked products produced by a fluidised catalytic cracking process.

An important advantage of the process according to the invention is that a high yield of C3-C5 olefins is obtained in the second reaction zone. It has been found that a feedstock obtained in a Fischer-Tropsch hydrocarbon synthesis process results in a much higher yield of C3-C5 olefins than a conventional FCC feedstock such as for example a vacuum gasoil (VGO) would produce under similar process conditions.

Another advantage is that the overall FCC process according to the invention is heat balanced, despite the use of a Fischer-Tropsch derived feedstock, since sufficient coke is deposited on the catalyst in the first reaction zone to balance for the lesser coke produced in the second reaction zone.

With respect to the process as disclosed in WO 2006/020547 an advantage of the process according to the invention is that the total amount of coke produced does not exceed the capacity of a single catalyst regenerator. Relatively severe cracking conditions can be used in the second reaction zone, resulting in a high yield of C3-C5 olefins, whilst not producing too much coke for the catalyst regenerator to remove.

Since at least the hydrocarbon feedstock to the second reaction zone is prepared by a Fischer-Tropsch hydrocarbon synthesis process, the process according to the invention can be advantageously integrated with the production of hydrocarbons from a hydrocarbonaceous feedstock such as natural gas or associated gas. In the production of hydrocarbons from a hydrocarbonaceous feedstock, the hydrocarbonaceous feedstock is first converted into synthesis gas, i.e. a gaseous mixture comprising carbon monoxide and hydrogen, and then the carbon monoxide and hydrogen are catalytically converted at elevated temperature and pressure into hydrocarbons by the so-called Fischer-Tropsch reaction.

An advantage of such integration is that off-gas from the hydrocarbon synthesis step or part of the hydrocarbonaceous feedstock may be used to provide the heat needed for endothermic process steps (a) and (c), in particular in case both the first and the second hydrocarbon feedstock comprise at least 70 wt % C₅+ hydrocarbons obtained in a Fischer-Tropsch process.

A still further advantage of such integration is that iso-butane needed for alkylation step (e) can be obtained by isomerising butane that will typically be co-produced with the hydrocarbonaceous feedstock from the same reservoir.

### SUMMARY OF THE DRAWINGS

FIG. 1 is a process flow scheme in accordance with the present invention.

### DETAILED DESCRIPTION OF THE INVENTION

In step (a) of the process according to the invention, a first hydrocarbon feedstock is catalytically cracked within a first reaction zone by contacting the first feedstock with a cracking catalyst comprising a shape-selective additive at a temperature in the range of from 450 to 650° C. to yield a first cracked product comprising middle distillate and a spent cracking catalyst.

The first reaction zone may comprise one or more riser or downcomer reactors, preferably one or more riser reactors.

The first feedstock may be a conventional hydrocarbon feedstock for catalytic cracking or a hydrocarbon stream obtained in a Fischer-Tropsch hydrocarbon synthesis process or a combination thereof. Preferably, the first hydrocarbon feedstock is a conventional hydrocarbon feedstock for catalytic cracking.

In the first reaction zone, a mixture of first cracked product and spent cracking catalyst is obtained. The mixture is separated, typically in a separator/stripper, in spent cracking catalyst and first cracked product. The first cracked product is preferably further separated into different streams in a separation system. A portion of the first cracked product, preferably a portion boiling in gasoline range, may be directed to the second reaction zone. A stream comprising middle distillates is recovered as product. The term “middle distillates”, as used herein, is a reference to hydrocarbon mixtures of which the boiling point range corresponds substantially to that of kerosene and gasoil fractions obtained in a conventional atmospheric distillation of crude mineral oil. The boiling point range of middle distillates generally lies within the range of 150 to 370° C.

A portion of the first cracked product comprising unconverted feedstock and/or HCO may be recycled to the first reaction zone. Preferably, a portion of the first cracked product comprising C₃₋₅ olefins is directed to the alkylation unit and alkylated therein, together with C₃₋₅ olefins from the second cracked product.

In step (b), the separated spent catalyst is regenerated to yield a regenerated cracking catalyst.

In step (c), at least part of the regenerated cracking catalyst obtained in step (b) is contacted, in a second reaction zone with a second hydrocarbon feedstock at a temperature in the range of from 500 to 800° C. to yield a second cracked product and a used regenerated catalyst. The second cracked product obtained in step (c) comprises gasoline and lower olefins such as ethylene, propylene and butylenes and minor amounts of compounds boiling above 232° C. Preferably, the second cracked product is separated into different fractions. More preferably, the second cracked product is separated into at least a fraction comprising ethylene and a fraction comprising C₃₋₅ olefins.

The second hydrocarbon feedstock comprises at least 70 wt % C₅₊ hydrocarbons obtained in a Fischer-Tropsch hydrocarbon synthesis process, preferably at least 90 wt %. Reference herein to hydrocarbons obtained in a Fischer-Tropsch hydrocarbon synthesis process is to a hydrocarbon stream obtained in the Fischer-Tropsch synthesis reaction, i.e. by catalytically converting carbon monoxide and hydrogen into hydrocarbons, or to a hydrocarbon stream that is obtained by hydroconversion of a hydrocarbon stream obtained by the Fischer-Tropsch synthesis reaction.

The process further comprises (step (d)) using regenerated catalyst obtained in step (c) as at least part of the cracking catalyst in step (a). Preferably, all used regenerated catalyst obtained in step (c) is used as at least part of the cracking catalyst in step (a). Preferably also part of the regenerated catalyst obtained in step (b) is used as part of the cracking catalyst in step (a).

In step (e) of the process according to the invention, at least a portion of the second cracked product is alkylated in an alkylation unit to obtain alkylate. The total second cracked product may be supplied to the alkylation unit. Preferably, a portion of the second cracked product predominantly comprising C₃₋₅ olefins is directed to the alkylation unit. In the alkylation unit, the C₃₋₅ olefins are reacted with an iso-paraffin such as iso-butane. This produces an iso-paraffin of higher molecular weight and improved octane rating compared to straight chain hydrocarbons. Generally, the alkylation of iso-paraffins with the olefins is accomplished by contacting the reactants with an acid catalyst such as hydrogen fluoride or sulphuric acid, settling the mixture to separate the catalyst from hydrocarbons, and further separating the hydrocarbons, usually by fractionation to recover the alkylate. Preferably, also a portion of the first cracked product comprising C₃₋₅ olefins is directed to the alkylation unit and the C₃₋₅ olefins therein are alkylated, together with the C₃₋₅ olefins from the second cracked product.

The first hydrocarbon feedstock preferably boils in the gasoil boiling point range or higher, i.e. in the range of from 210 to 750° C., more preferably above the gasoil boiling range, i.e. of from 350 to 650° C.

The second reaction zone may comprise a dense phase reactor, a fast fluidised reactor, a down-flow reactor, a fixed fluidized bed reactor, a riser reactor or a combination of said reactors. Preferably, the second reaction zone comprises a riser reactor or a fast fluidised bed reactor, more preferably a fast fluidised bed reactor.

Various factors affect whether a reactor is classified as a “fast fluidised reactor” particularly the gas velocity but also particle size, mean particle size, size distribution, particle density, solids flux rate and the size of the equipment. Herein a fast fluidised reactor is defined as a reactor with a gas velocity of 2-15 m/s, preferably 2-10 m/s, especially 3-5 m/s. A fast fluidised reactor typically comprises a strong density gradient along the vertical direction of the reactor. A dense region is provided in the bottom of a catalyst bed within the reactor (typically over 150 kg/m³ preferably over 200 kg/m³ for fluidized catalytic cracking), an extended transition region from dense to dilute is provided in the middle of the reactor and an extended dilute region in the top of the reactor. Preferably the dilute region is less than 100 kg/m³ for fluidized catalytic cracking catalyst, more preferably less than 50 kg/m³ especially less than 30 kg/m³.

Another suitable reactor for the second reaction zone is a dense phase reactor. The dense phase reactor can be a vessel that defines two zones, including a cracking or dense phase reaction zone, and a stripping zone. Contained within the cracking reaction zone of the vessel is cracking catalyst that is fluidized by the introduction of the feedstock.

In the second reaction zone, the feedstock is contacted with the catalyst at a temperature in the range of from 500 to 800° C., preferably of from 565° C. to 750° C., i.e. under relatively high severity cracking conditions, either with or without steam, to provide for a high yield of lower olefins. Apart from lower olefins, i.e. C₂-C₅ olefins, the second cracked product comprises unconverted gasoline plus minor amounts of higher boiling material.

The pressure within the second reaction zone can be up to 10 bar (absolute), preferably in the range of from 1.5 to 8.0 bar (absolute), more preferably of from 2.0 to 6.0 bar (absolute).

One way of controlling the operation of the second reaction zone is by the introduction of steam along with the feedstock. While the introduction of steam along with the feedstock is optional, a preferred aspect of the invention, however, is for steam to be introduced into the stripping zone of the reactor(s) in the second reaction zone and to be contacted with the cracking catalyst contained therein and in the cracking reaction zone. A preferred way of adding steam is by dividing the reactor into a lower and a higher zone. Introduce steam in the lower zone. Catalyst and feedstock are introduced in the higher zone; steam and hydrocarbon vapours are withdrawn from the top of the higher zone and catalyst from the bottom of the lower zone. The lower zone will then act to steam-strip the catalyst before it leaves the reactor, while the upper zone is primarily for reaction purposes. The use of steam in this manner provides, for a given conversion, an increase in the propylene yield and butylene yield.

If steam is added, at least 1 wt % steam is added to the second reaction zone, preferably at least 5 wt % steam, more preferably at least 8 wt % preferably, even more preferably in the range of from 10 to 30 wt %. The steam is preferably saturated steam or superheated steam.

Preferably the feed rate of catalyst/feedstock into the second reaction zone is less than 50, more preferably less than 30, especially less than 20.

Preferred catalytic cracking catalysts for the process according to the invention include fluidisable cracking catalysts comprised of a molecular sieve having cracking activity dispersed in a porous, inorganic refractory oxide matrix or binder. Molecular sieves suitable for use as a component of the cracking catalyst include pillared clays, delaminated clays, and crystalline aluminosilicates. Normally, it is preferred to use a cracking catalyst that contains a crystalline aluminosilicate. Examples of such aluminosilicates include Y zeolites, ultrastable Y zeolites, X zeolites, zeolite beta, zeolite L, offretite, mordenite, faujasite, and zeolite omega. The preferred crystalline aluminosilicates for use in the cracking catalyst are X and Y zeolites with Y zeolites being the most preferred.

The zeolite or other molecular sieve component of the cracking catalyst is typically combined with a porous, inorganic refractory oxide matrix or binder to form a finished catalyst prior to use. The refractory oxide component in the finished catalyst may be silica-alumina, silica, alumina, natural or synthetic clays, pillared or delaminated clays, mixtures of one or more of these components and the like. Preferably, the inorganic refractory oxide matrix will comprise a mixture of silica-alumina and a clay such as kaolin, hectorite, sepiolite and attapulgite. A preferred finished catalyst will typically contain between 5 and 40 weight percent zeolite or other molecular sieve and more than 20 weight percent inorganic, refractory oxide.

Preferably, the catalyst is a middle distillate selective cracking catalyst comprising amorphous silica alumina and a zeolite as molecular sieve having cracking activity.

The catalyst further comprises a shape-selective additive, which has high hydrothermal stability and a good selectivity towards producing olefins. A shape-selective additive further cracks C₅-C₈ olefins as produced in the second reaction zone to C₃ and C₄ olefins. A shape-selective additive also helps to increase branched hydrocarbons and aromatic content which increases gasoline octane rating. When a shape selective additive is used along with the middle distillate selective cracking catalyst in the second reaction zone, a huge improvement in the yield of propylene and butylenes can be achieved. Preferably, the catalyst comprises in the range of from 1 to 30 wt % of a shape-selective additive, preferably of from 3 to 20 weight percent, more preferably of from 5 to 18 weight percent.

The shape-selective additive may be embedded into catalyst before the catalyst is provided within the process or alternatively may be added to the process and allowed to contact the catalyst.

The shape-selective additive may be added to the regenerator or to one of the reactors of the second reaction zone if that reactor is a riser or a downcomer reactor. In case of a fast fluidised bed or a dense bed reactor in the second reaction zone, it is preferred to introduce the additive into the second reaction zone, along or concurrently with the regenerated cracking catalyst.

The shape-selective additive typically is a molecular sieves, preferably a medium pore zeolite. The medium pore size zeolites that can suitable be used as shape-selective additive generally have a pore size from about 0.5 nm, to about 0.7 nm and include, for example, MFI, MFS, MEL, MTW, EUO, MTT, HEU, FER, and TON structure type zeolites (IUPAC Commission of Zeolite Nomenclature). Non-limiting examples of such medium pore size zeolites, include ZSM-5, ZSM-12, ZSM-22, ZSM-23, ZSM-34, ZSM-35, ZSM-38, ZSM-48, ZSM-50, silicalite, and silicalite 2. The most preferred is ZSM-5, which is described in U.S. Pat. Nos. 3,702,886 and 3,770,614. Other suitable molecular sieves include the silicoaluminophosphates (SAPO), such as SAPO-4 and SAPO-11 which is described in U.S. Pat. No. 4,440,871; chromosilicates; gallium silicates, iron silicates; aluminium phosphates (ALPO), such as ALPO-11 described in U.S. Pat. No. 4,310,440; titanium aluminosilicates (TASO), such as TASO-45 described in EP-A No. 229,295; boron silicates, described in U.S. Pat. No. 4,254,297; titanium aluminophosphates (TAPO), such as TAPO-11 described in U.S. Pat. No. 4,500,651; and iron aluminosilicates. U.S. Pat. No. 4,368,114 describes in detail the class of zeolites that can be suitable shape-selective additives in the present invention.

The shape-selective additive may be held together with a catalytically inactive inorganic oxide matrix component, in accordance with conventional methods.

In the second reaction zone, the catalyst is conditioned so that when it is used for cracking of the first feedstock in the first reaction zone, the conditions are suitable for the production of a middle distillate product.

The second reaction zone can be operated or controlled independently from the operation or control of the first reaction zone. This independent operation or control of the second reaction zone provides the benefit of an improved overall control, i.e. across the first reaction zone and the second reaction zone, of the conversion of the feedstock into the desired end-products of middle distillate and the lower olefins, especially propylene and butylene. With the independent operation of the reaction zones, the severity of the first reaction zone cracking conditions can be reduced to thereby provide for a higher yield of middle distillate product in the cracked product, and the severity of the second reaction zone can be controlled to optimise the yield of olefins, in particular C₃-C₅ olefins.

Preferably, the process according to the present invention uses a middle distillate selective cracking catalyst in combination with steam addition to the second reaction zone, to provide for improved yields across the process system of middle distillate and C₃-C₅ olefins. In much of the prior art, it has generally been understood that in conventional reactor cracking processes low severity reactor cracking conditions result in less lower olefins yield relative to high severity gas oil reactor cracking conditions. The present invention, however, allows for the reduction in first reaction zone cracking severity in order to enhance the yield of middle distillate product while still providing for an increased yield in lower olefins via the use of the second reaction zone. The preferred use of steam in the second reaction zone provides further enhancements in the yield of lower olefins therefrom.

In the process, used regenerated cracking catalyst is removed from the second reaction zone and utilised as hot cracking catalyst mixed with the first feedstock that is introduced into the first reaction zone. One beneficial aspect of the present invention, in addition to its high yield of lower olefins, is that it provides for the partial deactivation of the catalyst prior to its use as hot cracking catalyst in the first reaction zone. What is meant by partial deactivation is that the used regenerated cracking catalyst will contain a slightly higher concentration of carbon than the concentration of carbon that is on a regenerated cracking catalyst. This partial deactivation of the cracking catalyst helps provide for an improved middle distillate product yield when the feedstock is cracked within the first reaction zone. The coke concentration on the used regenerated cracking catalyst is greater than the coke concentration on the cracking catalyst, but it is less than that of the separated spent cracking catalyst.

Another benefit of the process of the present invention is associated with the used regenerated cracking catalyst having a temperature that is lower than the temperature of the regenerated cracking catalyst. This lower temperature of the used regenerated cracking catalyst in combination with the partial deactivation, as discussed above, provides further benefits in a preferentially producing middle distillates from the cracking of the feedstock in the first reaction zone.

The combination of one or more of the above described process variables and operating conditions allows for the control of the conversion of the feedstock. Generally, it is desired for the first feedstock conversion to be in the range of from 40 to 98 wt %, preferably of from 50-90 wt %.

The mixture of feedstock and hot cracking catalyst, and, optionally, lift gas or steam, passes through the first reaction zone wherein cracking takes place. The first reaction zone defines a catalytic cracking zone and provides means for providing a contacting time to allow the cracking reactions to occur. The average residence time of the hydrocarbons in the first reaction zone generally can be up to 10 seconds, but usually is in the range of from 0.1 to 5 seconds. The weight ratio of catalyst to hydrocarbon feed (catalyst/oil ratio) generally can be in the range of from 2 to 100. More typically, the catalyst-to-oil ratio can be in the range of from 3 to 50, preferably of from 5 to 20.

The pressure within the first reaction zone may be up to 10 bar (absolute), preferably of from 1.5 to 8.0 bar (absolute), more preferably of from 2.0 to 6.0 bar (absolute).

The temperature in the first reaction zone is in the range of from about 450° C. to about 650° C., preferably in the range of from 480° C. to 560° C. The first reaction zone temperatures of the present invention will tend to be lower than those of typical conventional fluidised catalytic cracking processes, because the present invention is to provide for a high yield of middle distillates as opposed to the production of gasoline as is often sought with conventional fluidised catalytic cracking processes. Indeed, as more fully described elsewhere herein, one of the embodiments of the present invention provides for the control of certain of the process conditions within the first reaction zone by adjusting the ratio of regenerated cracking catalyst from the catalyst regenerator to used regenerated cracking catalyst from the second reaction zone that is introduced into the first reaction zone.

The mixture of hydrocarbons and catalyst from the first reaction zone pass as a first reaction zone product comprising cracked product and spent cracking catalyst to a stripper system that provides means for separating hydrocarbons from catalyst and defines a stripper separation zone wherein the cracked product is separated from the spent cracking catalyst. The stripper system can be any system or means known to those skilled in the art for separating catalyst from a hydrocarbon product. In a typical stripper operation, the first reaction zone product, which is a mixture of cracked product and spent cracking catalyst passes to the stripper system that includes cyclones for separating the spent cracking catalyst from the vaporous cracked product. The separated spent cracking catalyst enters the stripper vessel from the cyclones where it is contacted with steam to further remove cracked product from the spent cracking catalyst.

In step (b), the spent cracking catalyst is regenerated to yield a regenerated catalyst. As is conventional, the catalyst may be regenerated by combusting coke deposits thereon. The heat generated is typically exchanged with the reactor(s) in the first or second reaction zone (which are endothermic processes).

In case the first reaction zone is a riser reaction zone, lift gas or lift steam may also be introduced into the bottom of the first reaction zone along with the feedstock and the hot cracking catalyst.

Typically, the separated spent cracking catalyst is introduced in a regeneration zone wherein carbon that is deposited on the separated spent cracking catalyst is burned in order to remove the carbon to provide a regenerated cracking catalyst having a reduced carbon content. The catalyst regenerator typically is a vertical cylindrical vessel that defines the regeneration zone and wherein the spent cracking catalyst is maintained as a fluidized bed by the upward passage of an oxygen-containing regeneration gas, such as air.

The temperature within the regeneration zone is, in general, maintained in the range of from about 621° C. to 760° C., preferably in the range of from 677° C. to 715° C. The pressure within the regeneration zone typically is in the range of from atmospheric to 10 bar (absolute), preferably of from 1.5 to 8 bar (absolute), more preferably of from 2 to 6 bar (absolute). The residence time of the separated spent cracking catalyst within the regeneration zone is in the range of from 1 to 6 minutes, preferably of from 2 to 4 minutes.

The regenerated cracking catalyst that is yielded from the catalyst regenerator typically has a higher temperature than the used regenerated cracking catalyst that is yielded from the second reaction zone. Also, the used regenerated cracking catalyst has deposited thereon as a result of its use in the second reaction zone a certain amount of coke. A particular catalyst or combination of catalysts may be used to help control the conditions within the first reaction zone to provide for certain desired cracking conditions required to provide a desired product or mix of products.

The process according to the invention is preferably integrated with the production of hydrocarbons from a hydrocarbonaceous feedstock by a Fischer-Tropsch hydrocarbon synthesis process. Accordingly, the process preferably further comprises the following steps:

(i) converting a hydrocarbonaceous feedstock to a gaseous mixture comprising hydrogen and carbon monoxide;
(ii) catalytically converting the hydrogen and carbon monoxide at elevated temperatures and pressures to obtain normally gaseous, normally liquid and optionally normally solid hydrocarbons;
(iii) optionally hydrocracking and/or hydro-isomerising hydrocarbons obtained in step (ii) to obtain hydro-converted hydrocarbons;
wherein at least part of the hydrocarbons obtained in step (ii) and optionally step (iii), are used as the second hydrocarbon feedstock in step (c).

The hydrocarbonaceous feedstock that is converted into a gaseous mixture comprising hydrogen and carbon monoxide in step (i), may be a gaseous or solid hydrocarbonaceous feedstock. Preferably, the hydrocarbonaceous feedstock is a hydrocarbon gas, for example methane, natural gas, associated gas or a mixture of C₁₋₄ hydrocarbons. Alternatively, the feedstock may be a solid feedstock, for example coal, biomass, residuum from crude oil distillation, or tar-sand-derived bitumen.

Conversion step (i) may be any known process for the conversion of a hydrocarbonaceous feedstock into synthesis gas, typically a partial oxidation, autothermal reforming or steam reforming process. An example of a suitable partial oxidation process is the Shell Gasification Process. A comprehensive survey of this process can be found in the Oil and Gas Journal, Sep. 6, 1971, pp 86-90.

In step (i), a gaseous mixture comprising predominantly hydrogen and carbon monoxide is formed. Such mixture is typically referred to as synthesis gas. The mixture may contain nitrogen, carbon dioxide and/or steam.

In Fischer-Tropsch hydrocarbon synthesis step (ii), the synthesis gas is contacted with a suitable catalyst, and hydrocarbons are formed. The Fischer-Tropsch hydrocarbon synthesis is typically carried out at a temperature in the range of from 125 to 350° C., preferably of from 175 to 275° C., more preferably of from 200 to 260° C. The pressure preferably ranges of from 5 to 150 bar (absolute), more preferably of from 5 to 80 bar (absolute).

Hydrocarbons formed in step (ii) may range from methane to heavy paraffin waxes. Preferably, the production of methane is minimised and a substantial portion of the hydrocarbons produced have a carbon chain length of a least 5 carbon atoms. Preferably, the amount of C₅₊ hydrocarbons is at least 60% by weight of the total product, more preferably, at least 70% by weight, even more preferably, at least 80% by weight, most preferably at least 85% by weight. Usually the hydrocarbons formed are paraffinic of nature, while up to 30 wt %, preferably up to 15 wt %, of either olefins or oxygenated compounds may be present.

Optionally, all or part of the hydrocarbons obtained in step (ii) are hydrocracked and/or hydro-isomerised to obtain hydro-converted hydrocarbons.

At least part of the hydrocarbons obtained in step (ii) and/or the of hydroconverted hydrocarbons obtained in step (iii) are used as the second hydrocarbon feedstock in catalytic cracking step (c) as hereinbefore described. It is possible to also use part of the hydrocarbons obtained in step (ii) and/or of the hydroconverted hydrocarbons obtained in step (iii) as the first hydrocarbon feedstock in catalytic cracking step (a) as hereinbefore described.

If step (iii) is a hydrocracking step, the heavier molecules removed from the hydrocracker (“Hydrocracker Bottoms”) may also be used as a feedstock for catalytic cracking steps (a) and (c) according to the present invention, preferably for step (a).

Preferably, the part of the hydrocarbons obtained in steps (ii) and/or (iii) that boil above the boiling point range of the so-called middle distillates is used as the first hydrocarbon feedstock, i.e. for catalytic cracking step (a).

Gaseous hydrocarbons obtained in step (ii), i.e. C₁-C₄ hydrocarbons, may be combusted to provide a portion of the energy required for catalytic cracking steps (a) or (c). This mitigates and can even solve the problem of the energy imbalance between the endothermic catalytic cracking reactor and the regenerator in case a hydrocarbon feedstock obtained by Fischer-Tropsch hydrocarbon synthesis is used in both the first and the second reaction zone. Alternatively, or additionally, part of the gaseous hydrocarbon feedstock of step (i) may be used to provide a portion of the energy required for catalytic cracking steps (a) or (c).

In alkylation step (e), an iso-paraffin such as iso-butane is needed. If the catalytic cracking and alkylation process according to the invention is integrated with synthesis gas manufacture and Fischer-Tropsch hydrocarbon synthesis, i.e. with steps (i), (ii) and optionally (iii), then the iso-butane can advantageously be obtained by isomerising butane that is obtained from the same reservoir as the gaseous hydrocarbonaceous feedstock, typically predominantly methane, that is converted in step (i). Accordingly, the integrated process according to the invention preferably further comprises:

producing a gaseous hydrocarbonaceous feedstock, preferably predominantly methane, and butane from a reservoir;

using the gaseous hydrocarbonaceous feedstock as the hydrocarbonaceous feedstock in step (i);

isomerising the butane to obtain iso-butane; and

using the iso-butane in alkylation step (e).

### DETAILED DESCRIPTION OF THE DRAWINGS

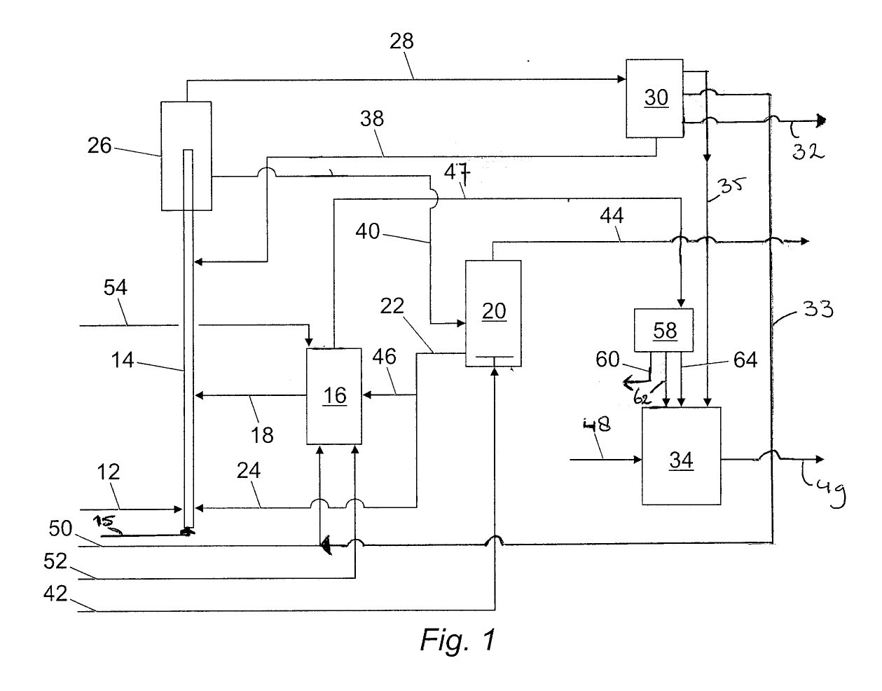

An embodiment of the present invention will now be described, by way of example only, with reference to FIG. 1.

FIG. 1 shows a process 10 comprising a first feedstock passing through conduit 12 and introduced into the bottom of catalytic cracking riser reactor 14.

In riser reactor 14, the first feedstock is mixed with a catalytic cracking catalyst. Steam may also be introduced into the bottom of riser reactor 14 by way of conduit 15. This steam can serve to atomize the feedstock or as a lifting fluid. The catalytic cracking catalyst can be a used regenerated cracking catalyst or a combination of used regenerated catalyst and regenerated catalyst. The used regenerated cracking catalyst is a regenerated cracking catalyst that has been used in a fast fluidised reactor 16 in the high severity cracking of a feedstock obtained by a Fischer-Tropsch process. The used regenerated cracking catalyst passes from fast fluidised reactor 16 and is introduced into riser reactor 14 by way of conduit 18. Regenerated cracking catalyst passes from regenerator 20 through conduit 22 and is introduced by way of conduit 24 into riser reactor 14 wherein it is mixed with the feedstock.

Passing through riser reactor 14 that is operated under catalytic cracking conditions is a feedstock obtained by a Fischer-Tropsch hydrocarbon synthesis process and hot catalytic cracking catalyst that forms a riser reactor product that comprises a mixture of a cracked product and a spent cracking catalyst. The riser reactor product passes from riser reactor 14 and is introduced into stripper system or separator/stripper 26.

The separator/stripper 26 can be any conventional system (such as a cyclonic separator) that defines a separation zone or stripping zone, or both, and provides means for separating the cracked product and spent cracking catalyst. The separated cracked product passes from separator/stripper 26 by way of conduit 28 to separation system 30. The separation system 30 can be any system known to those skilled in the art for recovering and separating the cracked product into the various products, such as, for example, cracked gas, cracked gasoline, cracked gas oils and cycle oil. The separation system 30 may include such systems as absorbers and strippers, fractionators, compressors and separators or any combination of known systems for providing recovery and separation of the products that make up the cracked product. A product stream comprising middle distillates is removed in line 32, a product stream comprising product boiling in the gasoline boiling range proceeds to the fast fluidised reactor 16 via line 33, a product stream comprising C₃-C₅ olefins is directed to alkylation unit 34 via line 35 and a bottom stream is recycled back to riser reactor 14 via line 38. The separated spent cracking catalyst passes from separator/stripper 26 through conduit 40 and is introduced into regenerator 20. Regenerator 20 defines a regeneration zone and provides means for contacting the spent cracking catalyst with an oxygen-containing gas, such as air, under carbon burning conditions to remove carbon from the spent cracking catalyst. The oxygen-containing gas is introduced into regenerator 20 through conduit 42 and the combustion gases pass from regenerator 20 by way of conduit 44.

Heat produced by the combustion of the coke in the regenerator 20 is used to provide heat for the fast fluidised reactor 16 and riser reactor 14.

The regenerated cracking catalyst passes from regenerator 20 through conduit 22. As an optional feature of the present invention, the stream of regenerated cracking catalyst passing through conduit 22 may be divided into two streams with at least a portion of the regenerated catalyst passing from regenerator 20 through conduit 22 passing through conduit 46 to fast fluidised reactor 16 and with the remaining portion of the regenerated catalyst passing from regenerator 20 passing through conduit 24 to riser reactor 14. To assist in the control of the cracking conditions in riser reactor 14, the split between the at least a portion of regenerated cracking catalyst passing through conduit 46 and the remaining portion of regenerated cracking catalyst passing through conduit 24 can be adjusted as required.

Fast fluidised reactor 16 defines a second reaction zone and provides means for contacting a feedstock with the regenerated cracking catalyst. The second reaction zone is operated under high severity cracking conditions to preferentially crack the second feedstock to lower olefin compounds, such as ethylene, propylene, and butylenes. The cracked product passes from fast fluidised reactor 16 through conduit 47 to alkylation unit 34 where it is combined with butane or other small alkanes (received from conduit 48) to produce alkylate (withdrawn via conduit 49), i.e. branched hydrocarbons with a high octane number.

The used regenerated cracking catalyst passes from fast fluidised reactor 16 through conduit 18 and is introduced into riser reactor 14. The feedstock is introduced into the fast fluidised reactor 16 through conduit 50 and steam is introduced into the fast fluidised reactor 16 by way of conduit 52. The feedstock and steam are introduced into the fast fluidised reactor 16 so as to provide for a fluidised bed of the regenerated catalyst. ZSM-5 is added as shape-selective additive to the regenerated catalyst of fast fluidised reactor 16 or introduced into reactor 16 through conduit 54.

In one embodiment of the present invention, a portion of the cracked product passing from separation system 30 may be recycled and introduced into the fast fluidised reactor 16 by way of conduit 33. This recycling of the cracked product provides for an additional conversion across the overall process system of the feedstock to desirable lower olefins. The cracked product from the fast fluidised reactor 16 passes through conduit 47 passes to olefin separation system 58. The olefin separation system 58 can be any system known to those skilled in the art for recovering and separating the cracked product into lower olefin product streams. The olefin separation system 58 may include such systems as absorbers and strippers, fractionators, compressors and separators or any combination of known systems or equipment providing for the recovery and separation of the lower olefin products from a cracked product. Yielded from the separation system 58 are ethylene product stream 60, propylene product stream 62, and butylenes product stream 64. Streams 62 and 64 pass from the olefin separation system 58 to alkylation unit 34.

### EXAMPLES

The process according to the invention will be further illustrated by means of the following non-limiting examples.

### Example 1

### Comparative

In a riser reactor, a vacuum gasoil with an initial boiling point of 138° C. and a final boiling point of 605° C. was contacted with a cracking catalyst comprising 12 wt % ZSM-5 as shape-selective additive at a temperature of 593° C. The gas residence time in the riser reactor was 3 seconds. In different experiments, the catalyst/oil ratio was varied. Total conversion of the feed, coke yield, yields of C3, C4 and C5-olefins were determined.

### Example 2

### Invention

EXAMPLE 1 was repeated but now with a Fischer-Tropsch derived wax (waxy raffinate) with an initial boiling point of 335° C. and a final boiling point of 557° C. as feedstock.

The results of EXAMPLES 1 and 2 are given in the Table below.

### Results of EXAMPLES 1 and 2

| C/O ratio | 6 | 8 | 10 | 12 |
|---|---|---|---|---|
| VGO | | | | |
| conversion (wt %) | 77 | 81 | 83 | 86 |
| coke yield(wt %) | 5 | 6 | 12 | 13 |
| C3 olefin yield (wt %) | 14 | 14 | 13 | 13 |
| C4 olefin yield (wt %) | 11 | 10 | 9 | 8 |
| C5 olefin yield (wt %) | 5 | 4 | 3 | 2 |
| Waxy raffinate | | | | |
| conversion (wt %) | 98 | 98 | 98 | 98 |
| coke yield(wt %) | 1.2 | 1.2 | 1.3 | 1.3 |
| C3 olefin yield (wt %) | 24 | 23 | 22 | 21 |
| C4 olefin yield (wt %) | 23 | 21 | 21 | 20 |
| C5 olefin yield (wt %) | 14 | 13 | 12 | 11 |

EXAMPLE 2 is an example of the second reaction zone in the process according to the invention. EXAMPLE 2 shows that of the second reaction zone is fed with a feedstock obtained in a Fischer-Tropsch hydrocarbon synthesis process, the conversion is higher than with a conventional FCC feedstock such as VGO (see EXAMPLE 1). Also, the yield of C3-C5 olefins, i.e. the olefins that may be alkylated in an alkylation unit, is significantly higher. Coke yield is much lower when using a feedstock obtained in a Fischer-Tropsch hydrocarbon synthesis process.

1. A process for the preparation of alkylate and middle distillate, the process comprising:
(a) catalytically cracking a first hydrocarbon feedstock by contacting the feedstock with a cracking catalyst comprising a shape-selective additive at a temperature in the range of from 450 to 650° C. within a riser or downcomer reaction zone to yield a first cracked product comprising middle distillate and a spent cracking catalyst;
(b) regenerating the spent cracking catalyst to yield a regenerated cracking catalyst;
(c) contacting, within a second reaction zone, at least part of the regenerated cracking catalyst obtained in step (b) with a second hydrocarbon feedstock at a temperature in the range of from 500 to 800° C. to yield a second cracked product and a used regenerated catalyst, the second feedstock comprising at least 70 wt % C₅₊ hydrocarbons obtained in a Fischer-Tropsch hydrocarbon synthesis process;
(d) using the used regenerated catalyst as at least part of the cracking catalyst in step (a); and
(e) alkylating at least a portion of the second cracked product in an alkylation unit to obtain alkylate.

2. A process as claimed in claim 1, wherein the second feedstock comprises at least 90 wt % C₅₊ hydrocarbons obtained in a Fischer-Tropsch hydrocarbon synthesis process.

3. A process according to claim 1, wherein the first cracked product is separated into a fraction comprising middle distillate and a fraction comprising C₃-C₅ olefins, and wherein the fraction comprising C₃-C₅ olefins is alkylated in the alkylation unit of step (e).

4. A process according to claim 1, wherein a portion of the first cracked product comprising hydrocarbons boiling in the gasoline boiling range is directed to the second reaction zone.

5. A process according to claim 1, wherein part of the regenerated cracking catalyst obtained in step (b) is used as part of the cracking catalyst in step (a).

6. A process according to claim 1, wherein the second reaction zone comprises a riser reactor or a fast fluidised bed reactor.

7. A process according to claim 1, wherein the shape-selective additive is ZSM-5.

8. A process according to claim 1, wherein the temperature in the first reaction zone is in the range of from 480 to 560° C.

9. A process according to claim 1, wherein the temperature in the second reaction zone is in the range of from 565 to 750° C.

10. A process according to claim 1, wherein at least 5 wt % steam is added to the second reaction zone.

11. A process according to claim 1, the process further comprising the following steps:
(i) converting a hydrocarbonaceous feedstock to a gaseous mixture comprising hydrogen and carbon monoxide;
(ii) catalytically converting the hydrogen and carbon monoxide at elevated temperature and pressure to obtain normally gaseous, normally liquid and optionally normally solid hydrocarbons;
(iii) optionally hydrocracking and/or hydro-isomerising the hydrocarbons obtained in step (ii) to obtain hydro-converted hydrocarbons;
wherein at least part of the hydrocarbons obtained in step (ii) and optionally step (iii), are used as the second hydrocarbon feedstock in step (c).

12. A process according to claim 11, wherein gaseous hydrocarbons obtained in step (ii) are combusted to provide a portion of the energy required for steps (a) or (c).

13. A process according to claim 11, the process further comprising:
producing a gaseous hydrocarbonaceous feedstock and butane from a reservoir;
using the gaseous hydrocarbonaceous feedstock as the hydrocarbonaceous feedstock in step (i);
isomerising the butane to obtain iso-butane; and
using the iso-butane in alkylation step (e).
